(19) United States
(12) Patent Application Publication
(10) Pub. No.: US 2013/0011477 A1
(43) Pub. Date: Jan. 10, 2013

(54) Stable Pharmaceutical Composition of Imatinib

(75) Inventors: Bandi Parthasaradhi Reddy, Hyderabad (IN); Podili Khadgapathi, Hyderabad (IN); Pothireddy Venkateswar Reddy, Hyderabad (IN); Muppidi Vanaja Kumari, Hyderabad (IN)

(73) Assignee: HETERO RESEARCH FOUNDATION, Hyderabad (IN)

(21) Appl. No.: 13/637,165

(22) PCT Filed: Mar. 29, 2010
(86) PCT No.: PCT/IN10/00202

§ 371 Date: Sep. 25, 2012

(51) Int. Cl. A61K 9/32; A61J 3/10; A61P 35/00; A61K 31/506; A61P 35/02

(52) U.S. Cl. 424/465; 424/482; 514/252.18; 264/109

(57) ABSTRACT

The present invention relates to a compressed film-coated tablet comprising imatinib its pharmaceutically acceptable salts there of in an amount of more than 80% based on the total weight of the finished dosage form.

### FIELD OF THE INVENTION

The present invention relates to a compressed film-coated tablet comprising imatinib its pharmaceutically acceptable salts there of in an amount of more than 80% based on the total weight of the finished dosage form and a film coating on said tablet core is applied in an amount of 1 to 2% w/w of the tablet.

### BACKGROUND OF THE INVENTION

Imatinib mesylate is chemically 4-[(4-Methyl1-piperazinyl)methyl]-N-[4-methyl-3-[[4-(3-pyridinyl)-2-pyrimidinyl]amino]-phenyl]benzamide methanesulfonate and is used to treat chronic myelogenous leukemia (CML), gastrointestinal stromal tumors (GISTs) and other cancers. In this document the term “imatinib” will refer to the mono methane sulfonate salt of imatinib unless otherwise specified.

Imatinib mesylate is a protein-tyrosine kinase inhibitor that inhibits the bcr-abl tyrosine kinase, the constitutive abnormal tyrosine kinase created by the Philadelphia chromosome abnormality in CML. Imatinib inhibits proliferation and induces apoptosis in bcr-abl positive cell lines as well as fresh leukemic cells from Philadelphia chromosome positive chronic myeloid leukemia. Imatinib inhibits colony formation in assays using ex vivo peripheral blood and bone marrow samples from CML patients.

U.S. Pat. No. 6,894,051 discloses the novel crystalline form of the mono methane sulfonic acid addition salt of imatinib and its preparation process and designated the form as β-crystalline form.

U.S. Pat. No. 6,958,335 describes the use of imatinib or a pharmaceutically acceptable salt thereof for the manufacture of pharmaceutical compositions for use in the treatment of gastrointestinal stromal tumours.

U.S. Pat. No. 7,544,799 discloses a particular form of methane sulfonic acid addition salt of imatinib.

PCT application WO 03/090720 describes a tablet comprising a pharmacologically effective amount of imatinib or a pharmaceutically acceptable salt thereof in an amount from about 30% to 80% in weight of the active moiety based on the total weight of tablet and pharmaceutically acceptable excipient.

PCT application WO 01/47507 describes a pharmaceutical composition comprising imatinib mesylate in an amount of 22% W/W.

US patent application US 2009/0087489 describes a pharmaceutical composition, preferably tablet, containing about 23-29% W/W of imatinib mesylate which were prepared using dry granulation or direct compaction.

As it is a high drug loaded composition, the present inventors have come across several problems in preparing the tablets of imatinib with such a low quantity of excipients. Thus, still there is a need for commercially acceptable high drug load compositions of imatinib.

Surprisingly, the present inventors have obtained a film coated tablet with high drug load of imatinib or pharmaceutical acceptable salts thereof and the tablet is film coated with opadry brown/yellow having poly vinyl alcohol as main ingredient.

### OBJECTIVE OF THE INVENTION

Accordingly, the main objective of the invention is to provide a film coated tablet comprising imatinib or pharmaceutically acceptable salts thereof in an amount of more than 80% based on the total weight of the coated tablet and a film coating comprising poly vinyl alcohol applied to the tablet core in an amount of 1 to 2% w/w of the tablet.

Yet another objective of the present invention is to provide a process for the preparation of stable film coated tablet of imatinib or its pharmaceutically acceptable salts.

### SUMMARY OF THE INVENTION

Accordingly, the main embodiment of the present invention is to provide a film coated tablet comprising imatinib or pharmaceutically acceptable salts thereof in an amount of more than 80% based on the total weight of the coated tablet and a film coating comprising poly vinyl alcohol applied to the tablet core in an amount of 1 to 2% w/w of the tablet.

### DETAILED DESCRIPTION OF THE INVENTION

According to the present invention there is provided a film coated tablet comprising imatinib or its pharmaceutically acceptable salts in an amount of more than 80% based on the total weight of the coated tablet and a film coating comprising poly vinyl alcohol applied to the tablet core in an amount of 1 to 2% w/w of the tablet.

Preferably, the present invention containing imatinib or pharmaceutically acceptable salts thereof in an amount of about 85 to 98% based on the total weight of the composition.

More preferably, the present invention containing imatinib or pharmaceutically acceptable salts thereof in an amount of about 90 to 97% based on the total weight of the composition.

The film coating, in addition to polyvinyl alcohol may consists of other ingredients such as titanium dioxide, talc, lecithin (soy), black iron oxide, polyethylene glycol and red iron oxide/yellow iron oxide or mixtures thereof.

Preferably, imatinib is in the mono mesylate form.

In another aspect, the present invention provides a film coated tablet wherein the mono mesylate salt of imatinib is in crystalline form or amorphous form.

Preferably, the mono mesylate salt of imatinib is in amorphous, alpha or beta crystalline form.

More preferably, the mono mesylate salt of imatinib is in alpha or beta crystalline form.

Still more preferably, the mono mesylate salt of imatinib is in alpha crystalline form.

Preferably, the film coating applied to the tablet core is in an amount of 1.3 to 1.6% w/w of the tablet.

The film coated tablet composition of the invention may contain one or more additional excipients. These excipients may be selected from binders and lubricants.

Preferably, the lubricant is selected from sodium stearyl fumarate, magnesium stearate, zinc stearate, calcium stearate, stearic acid, talc, Glyceryl behenate and collidal silicon dioxide.

More preferably, the lubricant is selected from magnesium stearate and colloidal silicon dioxide.

In a preferred embodiment of the present invention, the lubricant present in an amount of 0.8-2.5% w/w based on the total weight of the tablet.

The preferable binder is selected from L-Hydroxy propyl cellulose, polyvinyl pyrrolidine, hydroxylpropyl methyl cellulose, hydroxylethyl cellulose and pre-gelatinized starch.

More preferable binder is selected from L-Hydroxy propyl cellulose and pre-gelatinized starch.

The film coated tablet composition of imatinib or its pharmaceutically acceptable salts may be prepared by direct compression, wet granulation or roll compaction.

Preferably, the film coated tablet of imatinib or pharmaceutically acceptable salts thereof may be prepared by wet granulation.

The wet granulation process may be carried out using the solvent selected from isopropyl alcohol, ethanol, mixture of isopropyl alcohol and water and mixture of ethanol and water.

Preferably, the wet granulation process may be carried out using isopropyl alcohol.

The present invention also provides a process for the preparation of film coated tablet composition of imatinib or pharmaceutically acceptable salts thereof comprising:

i) Granulating imatinib or its pharmaceutically acceptable salts with binder solution.

ii) Drying the granules and then lubricating the dried granules

iii) Compressing the blended granules of step (ii) to obtained tablets.

iv) Coating the tablets of step (iii) with the coating suspension comprising polyvinyl alcohol.

The present invention also provides a process for the preparation of stable pharmaceutical composition of imatinib or its pharmaceutically acceptable salts comprising:

i) Compacting Imatinib or its pharmaceutically acceptable salts, optionally along with one pharmaceutically acceptable excipient.

ii) Lubricating the compact of step (i) with atleast one lubricant.

iii) Compressing the blend of step (ii) to obtained tablets.

iv) Coating the tablets of step (iii) coating suspension comprising polyvinyl alcohol.

In one of the embodiment, the film coating may be carried out with 12% W/W suspension of opadry brown/yellow in water.

The film coating, in addition to polyvinyl alcohol may comprises other ingredients such as titanium dioxide, talc, lecithin (soy), black iron oxide, polyethylene glycol and red iron oxide/yellow iron oxide or mixtures thereof.

The coating bed temperature is in the range of 40-45° C.

The film coated tablet composition of the present invention comprising about 100 to 400 mg of imatinib or its pharmaceutically acceptable salts.

The following examples further exemplify the invention and are not intended to limit the scope of the invention.

### Example 1

| Ingredients | Quantity/Tablet (mg) |
|---|---|
| Imatinib mesylate equivalent to imatinib | 478.00 |
| base | |
| Isopropyl alcohol | q.s |
| Magnesium Stearate | 12.0 |
| Coat (12% W/W) | 7.35 |
| Total | 497.35 |

The process steps involved in the preparation of Imatinib tablets are given below:

i) Granulated imatinib with binder solution i.e., isopropyl alcohol and dried the granulated mass

ii) Lubricated the dried granules with magnesium stearate

iii) Compressed the lubricated blend of step (ii) into tablets.

iv) Coated the tablets of step (iii) with 12% solution of Opadry brown/yellow in water.

### Example 2

| Ingredients | Quantity/Tablet (mg) |
|---|---|
| Imatinib mesylate equivalent to imatinib | 478.00 |
| base | |
| Isopropyl alcohol | q.s |
| Magnesium Stearate | 8.0 |
| Coat (12% W/W) | 7.35 |
| Total | 493.35 |

The process steps involved in the preparation of Imatinib tablets are given below:

i) Granulated imatinib with binder solution i.e., isopropyl alcohol and dried the granulated mass

ii) Lubricated the dried granules with magnesium stearate

iii) Compressed the lubricated blend of step (ii) into tablets.

iv) Coated the tablets of step (iii) with 12% solution of Opadry brown/yellow in water.

### Example 3

| Ingredients | Quantity/Tablet (mg) |
|---|---|
| Imatinib mesylate equivalent to imatinib base | 478.00 |
| Isopropyl alcohol | q.s |
| Magnesium Stearate | 4.0 |
| Coat (12% W/W) | 7.35 |
| Total | 489.35 |

The process steps involved in the preparation of Imatinib tablets are given below:

i) Granulated imatinib with binder solution i.e., isopropyl alcohol and dried the granulated mass

ii) Lubricated the dried granules with magnesium stearate

iii) Compressed the lubricated blend of step (ii) into tablets.

iv) Coated the tablets of step (iii) with 12% solution of Opadry brown/yellow in water.

### Example 4

| Ingredients | Quantity/Tablet (mg) |
|---|---|
| Imatinib mesylate equivalent to imatinib base | 478.00 |
| L-Hydroxy Propyl Cellulose | 4.0 |
| Magnesium Stearate | 8.0 |
| Coat (12% W/W) | 7.35 |
| Total | 497.35 |

The process steps involved in the preparation of Imatinib tablets are given below:

i) Imatinib, L-Hydroxy propyl cellulose and 50% of the magnesium stearate were subjected to roller compaction.

ii) Compacted blend was lubricated with remaining 50% of magnesium stearate

iii) Compressed the lubricated blend of step (ii) into tablets.

iv) Coated the tablets of step (iii) with 12% solution of Opadry brown/yellow in water.

### Example 5

| Ingredients | Quantity/Tablet (mg) |
|---|---|
| Imatinib mesylate equivalent to imatinib base | 478.00 |
| Colloidal silicon dioxide | 4.0 |
| Magnesium Stearate | 8.0 |
| Coat (12% W/W) | 7.35 |
| Total | 497.35 |

The process steps involved in the preparation of Imatinib tablets are given below:

i) Imatinib was subjected to roller compaction.

ii) Compacted imatinib was blended with colloidal silicon dioxide and magnesium stearate.

iii) Compressed the blend of step (ii) into tablets.

iv) Coated the tablets of step (iii) with 12% solution of Opadry brown/yellow in water.

We claim:

1. A film coated tablet comprising imatinib or pharmaceutically acceptable salts thereof in an amount of more than 80% based on the total weight of the coated tablet and a film coating comprising poly vinyl alcohol applied to the tablet core in an amount of 1 to 2% w/w of the tablet.

2. A film coated tablet according to claim 1 wherein imatinib or pharmaceutically acceptable salt there of is present in an amount of 85-98% based on the total weight of the dosage form.

3. A film coated tablet according to claim 1 wherein imatinib or pharmaceutically acceptable salt there of is present in an amount of 90-97% based on the total weight of the dosage form.

4. A film coated tablet according to claim 1 wherein the film coating, in addition to polyvinyl alcohol may consists of other ingredients such as titanium dioxide, talc, lecithin (soy), black iron oxide, polyethylene glycol and red iron oxide/yellow iron oxide or mixtures thereof.

5. A film coated tablet according to claim 1 wherein the imatinib is in the mono mesylate form.

6. A film coated tablet according to claim 1 wherein the mono mesylate salt of imatinib is in amorphous or crystalline form.

7. A film coated tablet according to claim 1 wherein the mono mesylate salt of imatinib is in amorphous, alpha or beta crystalline form.

8. A film coated tablet according to claim 1 wherein the mono mesylate salt of imatinib is in alpha or beta crystalline form.

9. A film coated tablet according to claim 1 wherein the mono mesylate salt of imatinib is in alpha crystalline form.

10. A film coated tablet according to claim 1 wherein the film coating applied to the tablet core is in an amount of from 1.3 to 1.6% w/w of the tablet.

11. A film coated tablet according to claim 1 further comprises one or more pharmaceutically acceptable excipients.

12. A film coated tablet according to claim 11 wherein the pharmaceutically acceptable excipients are selected from binders and lubricants.

13. A film coated tablet to claim 11 wherein the lubricant is selected from sodium stearyl fumarate, magnesium stearate, zinc stearate, calcium stearate, stearic acid, talc, Glyceryl behenate and colloidal silicon dioxide.

14. A film coated tablet according to claim 11 wherein the lubricant is magnesium stearate and colloidal silicon dioxide.

15. A film coated tablet according to claim 11 wherein the lubricant is in an amount of 0.8 to 2.5% w/w based on the total weight of the tablet.

16. A film coated tablet according to claim 11 wherein the binder is selected from L-Hydroxy propyl cellulose, polyvinyl pyrrolidine, hydroxylpropyl methyl cellulose, hydroxylethyl cellulose and pre-gelatinized starch.

17. A film coated tablet according to claim 11 wherein the binder is selected from L-Hydroxy propyl cellulose and pre-gelatinized starch.

18. A film coated tablet according to any one of the preceding claims prepared by wet granulation or roller compaction.

19. A film coated tablet according to claim 18 wherein the wet granulation is carried out using solvents selected from isopropyl alcohol, ethanol, mixture of isopropyl alcohol and water and mixture of ethanol and water.

20. A film coated tablet according to claim 18 wherein the wet granulation is carried out using isopropyl alcohol.

21. A process for the preparation of a stable solid oral composition according to any one of the preceeding claims, comprises
i). Granulating imatinib or its pharmaceutically acceptable salts with binder solution.
ii). Drying the granules and then lubricating the dried granules
iii). Compressing the blended granules of step (ii) to obtained tablets.

22. A process for the preparation of a stable solid oral composition according to any one of the preceeding claims, comprises
i). Compacting Imatinib or its pharmaceutically acceptable salts, optionally along with atleast one pharmaceutically acceptable excipient.
ii). Lubricating the compact of step (i) with atleast one lubricant
iii) Compressing the blend of step (ii) to obtained tablets.

23. A film coated tablet according to any one of the preceding claims wherein the tablet is film coated with 12% coating suspension in water.

24. A film coated tablet according to claim 1 wherein the composition comprises about 100 to 400 mg of imatinib or its pharmaceutically acceptable salts.
